(19) United States
(12) Patent Application Publication
(10) Pub. No.: US 2004/0200147 A1
(43) Pub. Date: Oct. 14, 2004

(54) Seedling culture mat

(76) Inventors: Fan-Juo Chang, Tainan Hsien (TW); Chi-Guang Wu, Nan-Tou Hsien (TW)

Correspondence Address: TROP PRUNER & HU, PC, 8554 KATY FREEWAY, SUITE 100, HOUSTON, TX, 77024, US

(21) Appl. No.: 10/818,734

(22) Filed: Apr. 6, 2004

(30) Foreign Application Priority Data
Apr. 14, 2003 (TW) 092205791

(51) Int. Cl. A01G 25/00; A01H 5/00

(52) U.S. Cl. 47/79

(57) ABSTRACT

A seedling culture mat includes a fibrous face layer, an intermediate layer, and a barrier substrate. The intermediate layer underlies the face layer, and includes a fibrous body, a plurality of seeds provided on the fibrous body, and a nutritional component applied to the fibrous body. The barrier substrate underlies the intermediate layer to guide primary roots which protrude out of the seeds to extend substantially along the barrier substrate before the primary roots project out of the barrier substrate. Thus, the primary roots and secondary roots which develop from the primary roots can be kept within the seedling culture mat for a prolonged period of time to take up the nutritional component.

### CROSS-REFERENCE TO RELATED APPLICATION

This application claims priority of Taiwanese Application No. 092205791, filed on Apr. 14, 2003.

### BACKGROUND OF THE INVENTION

1. Field of the Invention

The invention relates to a seedling culture mat, more particularly to a seedling culture mat that can be applied to the surface of a predetermined area of land for planting purposes and that can ensure germination of seeds into seedlings even under adverse conditions.

2. Description of the Related Art

In the past, planting in an area of land was achieved by sowing seeds directly in the soil so that the seeds could germinate and grow after watering. Although such a method was suitable for planting in a flat piece of land, a number of problems had to be overcome when the method was applied to a slope due to ready loss of moisture content in the soil and soil erosion. Besides, the method was not applicable to large-area planting. Therefore, a germination or culture mat was developed. A conventional culture mat is in the form of a large carpet which can be spread over a piece of land. Referring to FIG. 1, a conventional culture mat 1 includes a substrate 11 disposed to be in contact with the soil, a surface layer 12 overlying the substrate 11, and seeds 10 distributed between the substrate 11 and the surface layer 12. It is noted that the seeds 10 are actually very tiny, and that FIG. 1 is an enlarged view to facilitate illustration of the seeds 10.

After the culture mat 1 is spread over the soil, the seeds 10 in the culture mat 1 can germinate and grow into seedlings by implementing a proper watering scheme. Since the substrate 11 and the surface layer 12 can retain water, loss of water to the soil can be retarded. At the same time, the surface layer 12 covering the seeds 10 help anchor the seeds 10 in the culture mat 1 so that the appearance and growth of the seedlings can be relatively uniform. It is therefore apparent that use of the mat 10 in planting obviates the need to sow seeds, increases the rate of growth, enhances the overall appearance of the seedlings, and is suitable for planting in a relatively large area of land.

With further reference to FIGS. 2 and 3, typically, the process for a seed 10 to grow into a seedling 103 begins with the absorption of water by the seed 10. When the seed 10 starts to grow, a radicle emerges from the seed 10 and breaks the seed coat (testa). The radicle grows downward into the soil and becomes a primary root 101. After the primary root 101 has grown to a certain extent, secondary roots 102 start to branch out from the primary root 101. During the initial stage of growth, nutrients for the seed 10 are supplied by the seed 10 itself. But after the primary root 101 has developed, water moisture and nutrients, such as inorganic salts, are absorbed from the soil through the primary root 101 and later also through the secondary roots 102.

It is apparent from the foregoing that whether the primary and secondary roots 101, 102 can absorb sufficient nutrients is a critical factor governing successful germination of the seeds 10 into the seedlings 103. While the conventional culture mat 1 can overcome the drawbacks associated with the direct sowing method, since the secondary roots 102 develop from the primary root 101 after penetration of the primary root 101 through the surface layer 12, when the culture mat 1 is used for planting in a barren spot, even if the primary root 101 can grow and extend into the soil, the secondary roots 102 which develop from the primary root 101 cannot obtain sufficient water and nutrients for growth, thereby resulting in withering of the plant.

### SUMMARY OF THE INVENTION

Therefore, the object of the present invention is to provide a seedling culture mat that can enable seeds to germinate into seedlings even under adverse conditions.

Accordingly, a seedling culture mat of this invention includes a fibrous face layer, an intermediate layer, and a barrier substrate. The intermediate layer underlies the face layer, and includes a fibrous body, a plurality of seeds provided on the fibrous body, and a nutritional component applied to the fibrous body. The barrier substrate underlies the intermediate layer to guide primary roots which protrude out of the seeds to extend substantially along the barrier substrate before the primary roots project out of the barrier substrate. As such, the primary roots and secondary roots which develop from the primary roots can be kept within the seedling culture mat for a prolonged period of time to take up the nutritional component.

### BRIEF DESCRIPTION OF THE DRAWINGS

Other features and advantages of the present invention will become apparent in the following detailed description of the preferred embodiment with reference to the accompanying drawings, of which:

FIG. 1 is a fragmentary schematic partly sectional view of a conventional culture mat in part;

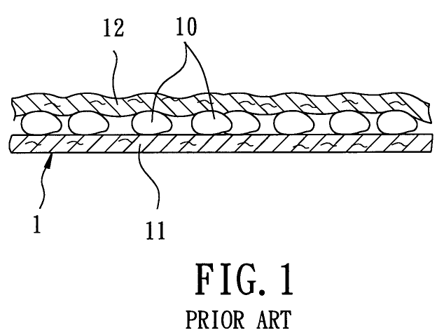

FIG. 2 is a schematic view to illustrate the germination of a seed in the conventional culture mat;

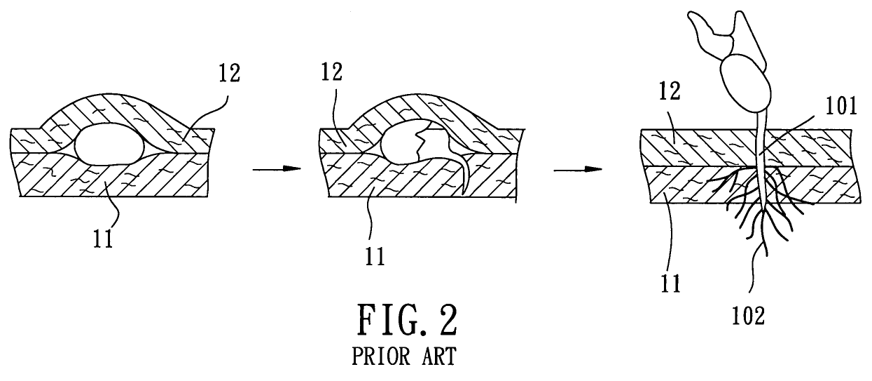

FIG. 3 illustrates a typical process of seed germination;

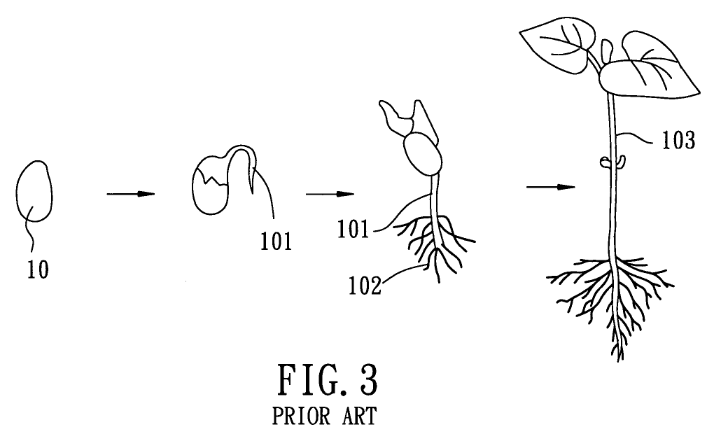

FIG. 4 is a fragmentary schematic partly sectional view illustrating the preferred embodiment of a seedling culture mat according to this invention; and

FIG. 5 is a schematic view illustrating the germination of a seed in the preferred embodiment.

### DETAILED DESCRIPTION OF THE PREFERRED EMBODIMENT

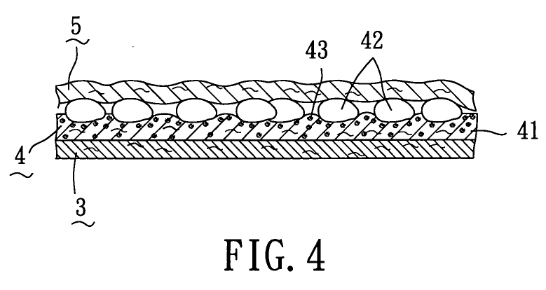

Referring to FIG. 4, the preferred embodiment of a seedling culture mat according to the present invention is shown to include a fibrous face layer 5, an intermediate layer 4, and a barrier substrate 3. Each of the fibrous face layer 5, the intermediate layer 4, and the barrier substrate 3 is made of a material selected from the group consisting of synthetic fibers and natural fibers, and from the group consisting of a non-woven fabric, rayon, nylon, woven fabric, and a knitted fabric. In this embodiment, each of the fibrous face layer 5, the intermediate layer 4, and the barrier substrate 3 is exemplified to be made of a non-woven fabric.

Preferably, the fibrous face layer 5 has a thickness of about 0.2 mm-1.0 mm, the intermediate layer 4 has a thickness of about 3 mm to 8 mm, and the barrier substrate 3 has a thickness of about 1 mm to 3 mm. In this embodiment, the fibrous face layer 5 has a thickness of 0.5 mm, the intermediate layer 4 has a thickness of 5 mm, and the barrier substrate 3 has a thickness of 2 mm. It is noted that this invention should not be limited to the thickness exemplified in the preferred embodiment.

The intermediate layer 4 underlies the fibrous face layer 5, and includes a fibrous body 41, a plurality of seeds 42 provided on the fibrous body 41, and a nutritional component 43 applied to the fibrous body 41. The seeds 42 are positioned on the fibrous body 41 by the fibrous face layer 5. The nutritional component 43 may be a mixture of a microbe, peat, and an organic fertilizer. The microbe may be any growth-promoting mycorrhizae, such as vesicular-arbuscular (VA) mycorrhizae, in powder form. According to the variety of the seeds, the nutritional component 43 may include a microbe only, i.e., without peat and fertilizer. In addition, the nutritional component 43 is applied to the fibrous body 41 by spraying. Since the fibrous body 41 has minute pores, the nutritional component 43 applied to the fibrous body 41 will not only stay on the surface of the fibrous body 41, but will also penetrate the fibrous body 41. As such, the nutritional component 43 will form a dense network of hyphae.

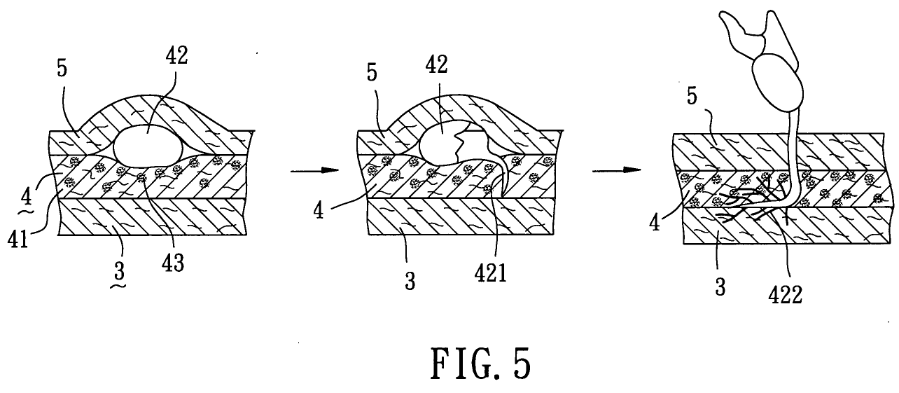

The barrier substrate 3 underlies the intermediate layer 4 to guide primary roots 421 which protrude out of the seeds 42 to extend substantially along the barrier substrate 3 before the primary roots 421 project out of the barrier substrate 3 so that the primary roots 421 and secondary roots 422 which develop from the primary roots 421 can be kept with in the seedling culture mat for a prolonged period of time to take up the nutritional component 43. Furthermore, the barrier substrate 3 is a fibrous substrate which has a density greater than that of the intermediate layer 4. As such, referring to FIG. 5, when the primary roots 421 protrude from the seeds 42, they are prevented from extending directly downward into the barrier substrate 3 below, and will extend substantially horizontally for a certain distance before extending obliquely and gradually into the barrier substrate 3. Since the primary roots 421 do not extend directly through the barrier substrate 3, the secondary roots 422 that develop therefrom will spread out between the intermediate layer 4 and the barrier substrate 3, and will be infected by the network of hyphae to thereby develop a symbiotic relationship therewith. Thus, even in a desolate and barren land, or in a soil environment with a pH ranging from 3 to 10, by means of the network of hyphae that help absorb nutrients and water moisture from the soil, the seeds 42 can grow into seedlings.

In summary, this invention utilizes the barrier substrate 3 to guide the direction of extension of the primary roots 421 so that the secondary roots 422 that subsequently grow from the primary roots 421 can be infected extensively by the growth-promoting microbe in the nutritional component 43, thereby increasing the rate of seedlings produced from the seeds 42.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

We claim:

1. A seedling culture mat comprising:
a fibrous face layer;
an intermediate layer underlying said face layer and including a fibrous body, a plurality of seeds provided on said fibrous body, and a nutritional component applied to said fibrous body; and
a barrier substrate underlying said intermediate layer to guide primary roots which protrude out of said seeds to extend substantially along said barrier substrate before the primary roots project out of said barrier substrate, whereby, the primary roots and secondary roots which develop from the primary roots can be kept within said seedling culture mat for a prolonged period of time to take up said nutritional component.

2. The seedling culture mat as claimed in claim 1, wherein said barrier substrate is a fibrous substrate which has a density greater than that of said intermediate layer.

3. The seedling culture mat as claimed in claim 2, wherein each of said barrier substrate, said intermediate layer and said face layer is made of a material selected from the group consisting of synthetic fibers and natural fibers.

4. The seedling culture mat as claimed in claim 2, wherein each of said barrier substrate, said intermediate layer and said face layer is made of a material selected from the group consisting of a non-woven fabric, a woven fabric, and a knitted fabric.

5. The seedling culture mat as claimed in claim 2, wherein said nutritional component includes a substance selected from the group consisting of a microbe and peat.

6. The seedling culture mat as claimed in claim 5, wherein said nutritional component includes a microbe and peat.

7. The seedling culture mat as claimed in claim 2, wherein said nutritional component includes a fertilizer.

8. The seedling culture mat as claimed in claim 5, wherein said nutritional component includes a microbe which stimulates plant growth.

9. The seedling culture mat as claimed in claim 8, wherein said microbe is vesicular-arbuscular mycorrhizae.

10. The seedling culture mat as claimed in claim 1, wherein said fibrous face layer has a thickness of about 0.2 mm-1.0 mm, said intermediate layer has a thickness of about 3 mm to 8 mm, and said barrier substrate has a thickness of about 1 mm to 3 mm.
